(19) United States
(12) Patent Application Publication
(10) Pub. No.: US 2019/0117552 A1
(43) Pub. Date: Apr. 25, 2019

(54) AQUEOUS EMULSIONS CONTAINING AMINO-FUNCTIONAL ORGANOPOLYSILOXANES AND NON-IONIC EMULSIFIERS

(71) Applicant: Wacker Chemie AG, Munich (DE)

(72) Inventors: Holger RAUTSCHEK, Nuenchritz (DE); Marco KAUSCHKE, Glaubitz (DE)

(73) Assignee: Wacker Chemie AG, Munich (DE)

(21) Appl. No.: 16/098,772

(22) PCT Filed: Apr. 5, 2017
(86) PCT No.: PCT/EP2017/058070

§ 371 Date: Nov. 2, 2018

(30) Foreign Application Priority Data
May 3, 2016 (DE) 10 2016 207 603.5

(51) Int. Cl. A61K 8/898; A61Q 5/02; A61Q 5/12; A61K 8/86; A61K 8/36; A61K 8/34; C11D 11/00; C11D 17/00; C11D 3/37; C11D 1/835; C11D 1/831

(52) U.S. Cl. CPC A61K 8/898; A61Q 5/02; A61Q 5/12; A61K 8/86; A61K 8/36; A61K 8/34; C11D 1/72; C11D 17/0017; C11D 3/3742; C11D 1/835; C11D 1/831; A61K 2800/21; C11D 11/0017

(57) ABSTRACT

Aqueous emulsions containing a high proportion of aminoalkyl-functional organopolysiloxane and a lesser proportion of emulsifying surfactant are stable and clear when the emulsifying surfactant comprises an ethoxylated linear alcohol having 10 carbon atoms or less.

### CROSS REFERENCE TO RELATED APPLICATIONS

This application is the U.S. National Phase of PCT Appln. No. PCT/EP2017/058070 filed Apr. 5, 2017, which claims priority to German Application No. 10 2016 207 603.5 filed May 3, 2016, the disclosures of which are incorporated in their entirety by reference herein.

### BACKGROUND OF THE INVENTION

### 1. Field of the Invention

The invention relates to aqueous emulsions containing amino-functional organopolysiloxanes and nonionic emulsifiers and to a process for the production thereof.

### 2. Description of the Related Art

Silicones have a multiplicity of uses. In order to facilitate use and dosing, in particular in viscous products, it is desirable in many applications for the organosilicon compounds to be in diluted form. They are therefore usually used in the form of aqueous emulsions or dispersions, typically as oil-in-water emulsions (O/W-emulsions) which are dilutable with water. EP 138192 A1 discloses that particularly finely divided emulsions, so-called microemulsions, which are translucent to clear may be produced based on organopolysiloxanes having polar groups. Such emulsions are advantageously employable in particular where exceptional stability of these emulsions is required, for example in textile processing where oil stains must be avoided or when, for example, clear cosmetic formulations are to be produced.

Proposed emulsifiers for producing these microemulsions are predominantly nonionic emulsifiers and in particular ethoxylated alcohols. Examples are ethoxylated trimethylnonanol (e.g. EP 299596 B1, EP 943644 B1), ethoxylated isotridecyl alcohols (e.g. EP 442098 B1, EP 515915 B1 and EP 859029 A2), ethoxylated isodecyl alcohols (e.g. EP 1560972 B1), mixtures of ethoxylated isotridecyl alcohols and ethoxylated oxo alcohols (i.e. branched alcohols) having 10 carbon atoms (DE 19835227 A1) and ethoxylated fatty alcohols such as n-dodecanol (e.g. EP 475363 A2). In addition to the emulsifiers the microemulsions may contain solvents, for example alcohols (e.g. EP 417559 B1), or glycols, such as monoethylene glycol hexyl ether, monoethylene glycol butyl ether, diethylene glycol hexyl ether, diethylene glycol butyl ether (e.g. WO15013247 A1), or polyols (e.g. EP 0859029 A2). These microemulsions normally contain not more than 20% of siloxane having polar groups (e.g. EP 2215148 B1). At higher proportions the viscosity increases severely so that the handling of the products becomes difficult to impossible (cf. DE 19835227 A1 page 3 lines 28-34).

DE 19835227 A1 discloses that by blending the amino-functional organopolysiloxanes with unsubstituted polydimethylsiloxane and addition of polyethylene glycol it is possible to obtain clear products having a siloxane proportion of over 30%; otherwise a firm gel is formed. Addition of salt to this gel reduced the viscosity but also the stability, and addition of isopropanol resulted in a product having a low flame point which is likewise undesirable.

In addition the viscosity may be controlled only to a limited extent by the addition of the abovementioned glycols. Thus for example in addition to 10% of emulsifier a further 15% of butyl diglycol are required to obtain a handleable microemulsion comprising 30% of amino-modified organopolysiloxane (EP 646618 B1), i.e. over 80% of emulsifiers/glycols based on the organopolysiloxane. This high proportion of constituents which do not contribute to the efficacy of the product in the application, but entail corresponding costs and ultimately also have an unnecessary environmental impact, is not desired.

### SUMMARY OF THE INVENTION

The problem addressed by the present invention is accordingly that of providing translucent to clear emulsions having a small particle size, based on amino-functional organopolysiloxanes which, even at a high content of organopolysiloxane, are readily handleable and storage-stable and contain no solvents.These and other problems are solved by the invention, which provides aqueous emulsions containing
(A) amino-functional organopolysiloxanes containing Si—C-bonded radicals comprising basic nitrogen,
(B) optionally organopolysiloxanes which are distinct from the amino-functional organopolysiloxanes (A),
(C) nonionic emulsifiers,
(D) optionally ionic emulsifiers,
(E) optionally auxiliaries and
(F) water,
   characterized in that as nonionic emulsifiers (C) polyethylene glycol ethers of linear primary alcohols comprising linear hydrocarbon radicals having not more than 10 carbon atoms are employed,
   with the proviso that the co-use of polyethylene glycol ethers of primary alcohols comprising branched hydrocarbon radicals and the co-use of polyethylene glycol ethers of secondary alcohols are excluded.

### DETAILED DESCRIPTION OF THE PREFERRED EMBODIMENTS

Organopolysiloxanes (A) are preferably organopolysiloxanes containing units of formula

Rₐ(R²O)_{b}Q_{c}SiO_{(4-a-b-c)/2} (I),

in which
R is identical or different and represents a monovalent, optionally substituted hydrocarbon radical having 1 to 30 carbon atoms or hydrogen,
R² is identical or different and represents hydrogen or a monovalent, optionally substituted hydrocarbon radical having 1 to 20, preferably 1 to 6 carbon atoms,
Q represents an amine-substituted radical of formula

—R³—[NR⁴—R⁵—]ₓNR⁴2 (II)

in which
R³ is identical or different and represents a divalent Si—C-bonded hydrocarbon radical having 1 to 18 carbon atoms,
R⁴ represents hydrogen or a monovalent linear, cyclic or branched, saturated or unsaturated hydrocarbon radical having 1-18 carbon atoms,
R⁵ is identical or different and represents a divalent hydrocarbon radical having 1 to 6 carbon atoms,
x is 0 or an integer from 1 to 10, preferably 0 or 1,
a is 0, 1, 2 or 3,
b is 0, 1, 2 or 3, preferably 0 or 1, and
c is 0, 1, 2 or 3, preferably 0 or 1,
   with the proviso that the sum of a+b+c is less than or equal to 3.

It is preferable when the sum of a+b+c has an average value of 1.5 to 2.5, preferably 1.9 to 2.1.

Examples of unsubstituted hydrocarbon radicals R include alkyl radicals such as the methyl, ethyl, n-propyl, iso-propyl, 1-n-butyl, 2-n-butyl, iso-butyl, tert-butyl, n-pentyl, iso-pentyl, neo-pentyl, and tert-pentyl radicals; hexyl radicals such as the n-hexyl radical; heptyl radicals such as the n-heptyl radical; octyl radicals such as the n-octyl radical and iso-octyl radicals such as the 2,2,4-trimethylpentyl radical; nonyl radicals such as the n-nonyl radical; decyl radicals such as the n-decyl radical; dodecyl radicals such as the n-dodecyl radical; octadecyl radicals such as the n-octadecyl radical; cycloalkyl radicals such as the cyclopentyl, cyclohexyl, cycloheptyl radicals and methylcyclohexyl radicals; alkenyl radicals such as the vinyl, 1-propenyl and 2-propenyl radicals; aryl radicals, such as the phenyl, naphthyl, anthryl and phenanthryl radicals; alkaryl radicals such as o-, m-, p-tolyl radicals, xylyl radicals, and ethylphenyl radicals; and aralkyl radicals, such as the benzyl radical and the α- and β-phenylethyl radicals.

Examples of substituted radicals R include halogen-, cyano-, glycidoxy- or polyalkylene glycol-substituted hydrocarbon radicals, for example trifluoropropyl, cyanoethyl, glycidoxypropyl, polyalkylene glycol propyl radicals.

Preferred hydrocarbon radicals R are methyl and phenyl radicals.

Examples of radicals R² include the hydrogen atom and methyl, ethyl, propyl and butyl radicals. It is preferable when the radical R² is a hydrogen atom, an ethyl radical or a methyl radical.

Examples of amine-substituted radicals Q include:
H₂N(CH₂)₂NH(CH₂)₃—
H2N(CH₂)₃—
H₂N(CH₂)₂NH—CH₂—CH(CH₃)—CH₂—
H₂N—CH₂—CH(CH₃)—CH₂—
H₃CNH(CH₂)₃—
H₂N(CH₂)₄—
H₂N(CH₂)₅—
H(NHCH₂CH₂)₃—
C₄H₉NH(CH₂)₂NH(CH₂)₂— and
cyclo-C₆H₁₁NH(CH₂)₃—,
wherein
H₂N(CH₂)₃— and
H₂N(CH₂)₂NH(CH₂)₃— are particularly preferred.

The organopolysiloxanes (A) used in the process according to the invention are preferably those containing 5 to 10,000, in particular 50-1000, units of formula (I), more preferably those composed of units of formula (I) having an average value of a of 1.90 to 2.3, an average value of b of 0 to 0.2, an average value of c of 0.002 to 0.1.

It is preferable when the organopolysiloxanes (A) contain no further substituted hydrocarbon radicals other than the amine-substituted radicals R².

Preferred organopolysiloxanes (A) are those of formula

R*_{3-y}Q_{y}SiO[R₂SiO]ₖ[RQSiO]₁SiQ_{y}R*_{3-y} (III),

wherein
R* represents R or a radical of formula —OR²,
R, R² and Q are as defined above,
k is an integer from 10 to 1000,
l is an integer from 1 to 100 and
y is 0 or 1, preferably 0.

The organopolysiloxanes (A) preferably have a viscosity of 50 mm²/s to 100,000 mm²/s measured at 25° C. according to DIN 53019.

The organopolysiloxanes (A) preferably have a content of basic nitrogen of 0.05 mmol/g to 2.0 mmol/g, particularly preferably 0.15 mmol/g to 1.0 mmol/g.

Optionally co-used organopolysiloxanes (B) preferably consist of units of formula

R⁸ₑ(R⁹O)_{f}SiO_{(4-e-f)/2} (IV),

wherein
R⁸ is identical or different and represents a monovalent, optionally substituted hydrocarbon radical having 1 to 30 carbon atoms or a hydrogen atom,
R⁹ is identical or different and represents a hydrogen atom or a monovalent, optionally substituted hydrocarbon radical having 1 to 20, preferably 1 to 6, carbon atoms,
e is 0, 1, 2 or 3 and
f is 0, 1, 2 or 3, preferably 0 or 1.

Examples of radicals R are fully applicable to radicals R⁸.

Examples of radicals R² are fully applicable to radicals R⁹.

The organopolysiloxanes (B) are preferably polydimethylsiloxanes having trimethylsilyl end groups or dimethylhydroxysilyl end groups.

The organopolysiloxanes (B) preferably have a viscosity of 5 mm²/s-1000 mm²/s measured at 25° C. according to DIN 53019.

It is preferable when the co-use of organopolysiloxanes of formula

R¹(CH₃)₂SiO[(CH₃)₂SiO]ₙSi(CH₃)₂R¹ (V),
wherein R¹ represents a hydroxyl, methoxy, ethoxy or methyl radical and n represents 25 to 900,
in amounts of greater than or equal to 2% by weight, preferably greater than 1% by weight, in each case based on the total weight of the emulsion, is excluded.

It is particularly preferable when the co-use of polyorganosiloxanes (B) which are distinct from the amino-functional organopolysiloxanes (A) in amounts of greater than or equal to 2% by weight, preferably greater than 1% by weight, in each case based on the total weight of the emulsion, is excluded.

In a particularly preferred embodiment the emulsions according to the invention contain no organopolysiloxanes (B).

The polyethylene glycol ethers (C) are preferably linear primary alcohols having not more than 10 carbon atoms those of formula

CH_{3—}(CH₂)ₘ—O—(CH_{2—}CH_{2—}O)ₚ—R⁶ (VI),
wherein R⁶ represents a hydrogen atom, a C₁₋₄-hydrocarbon radical or a radical of formula —C(O)R⁷,
R⁷ represents a C₁₋₂-hydrocarbon radical
m represents an integer from 5 to 9 and
p is an integer from 3 to 20.
R⁶ is preferably a hydrogen atom.

Examples of nonionic emulsifiers (C) according to the invention are polyethylene glycol ethers of formula:

CH_{3—}(CH₂)₅—O—(CH_{2—}CH_{2—}-O)₄—H

CH_{3—}(CH₂)₅—O—(CH_{2—}CH_{2—}O)₅—H

CH_{3—}(CH₂)₇—O—(CH_{2—}CH_{2—}O)₄—H

CH_{3—}(CH₂)₇—O—(CH_{2—}CH_{2—}O)₅—H

CH_{3—}(CH₂)₇—O—(CH_{2—}CH_{2—}O)₆—H

CH_{3—}(CH₂)₇—O—(CH_{2—}CH_{2—}O)₈—H

CH_{3—}(CH₂)₉—O—(CH_{2—}CH_{2—}O)₅—H

CH_{3—}(CH₂)₉—O—(CH_{2—}CH_{2—}O)₆—H

CH_{3—}(CH₂)₉—O—(CH_{2—}CH_{2—}O)₁₀—H

CH_{3—}(CH₂)₉—O—(CH_{2—}CH_{2—}O)₁₆—H

CH_{3—}(CH₂)₉—O—(CH_{2—}CH_{2—}O)₂₀—H

It is preferable when the co-use of polyethylene glycol ethers of branched primary alcohols, i.e. those comprising branched hydrocarbon radicals, and the co-use of polyethylene glycol ethers of secondary alcohols, is excluded.

It is preferable when the emulsions according to the invention contain no (poly)ethylene glycols or (poly)ethylene glycol ethers with alcohols having less than 6 carbon atoms, alkylene glycols, glycerol, monovalent alcohols having up to 5 carbon atoms or polyvalent alcohols having up to 10 carbon atoms.

The emulsions according to the invention preferably contain no emulsifiers based on ethoxylated alkylphenols.

The co-use of further nonionic emulsifiers which are distinct from the nonionic emulsifiers (C) according to the invention is preferably excluded.

Examples of optionally present ionic emulsifiers (D) include in particular cationic emulsifiers.

Examples of cationic emulsifiers (D) include all known quaternary ammonium compounds bearing at least one substituted or unsubstituted hydrocarbon radical having at least 10 carbon atoms, such as dodecyldimethylammonium chloride, tetradecyltrimethylammonium bromide, stearyltrimethylammonium chloride, distearyldimethylammonium chloride, cetyltrimethylammonium chloride, behenyltrimethylammonium bromide, dedecylbenzyldimethylammonium chloride and benzyltrimethylammonium chloride. Ethoxylated cationic emulsifiers may also be employed, for example cocoalkylbis(hydroxyethyl)methyl, ethoxylated, methylsulfate.

When cationic emulsifiers are employed as component (D), these are preferably aryl- or alkyltrimethylammonium salts, such as stearyltrimethylammonium chloride and cetyltrimethylammonium chloride, benzyltrialkylammonium salts, trimethylbenzylammium chloride and trimethylbenzylammonium methosulfate.

Further examples include all known quaternary imidazolinium compounds bearing at least one substituted or unsubstituted hydrocarbon radical having at least 10 carbon atoms, such as 1-methyl-2-stearyl-3-stearylamidoethylimidazolinium methosulfate, 1-methyl-2-norstearyl-3-stearylamidoethylimidazolinium methosulfate, 1-methyl-2-oleyl-3-oleylamidoethylimidazolinum methosulfate, 1-methyl-2-stearyl-3-methylimidazolinum methosulfate, 1-methyl-2-behenyl-3-methylimidazolinum methosulfate and 1-methyl-2-dodecyl-3-methylimidazolinum methosulfate.

Employable as auxiliaries (E) are all substances which are typically added to silicon emulsions and are distinct from the components (A), (B), (C), (D) and (F), for example silanes, in particular alkoxysilanes, rheology additives, preservatives, pH regulators, disinfectants, wetting agents, corrosion inhibitors, colorants and fragrances.

Examples of auxiliaries (E) include in particular preservatives, for example benzyl alcohol, phenoxyethanol, parabens, salicylic acid, isothiazolinones and pH regulators, for example formic acid, acetic acid, propionic acid and salts thereof with alkali metals or alkaline earth metals or amines.

Employable as water (F) are all types of water that have hitherto also been used for producing dispersions.

Preferably employed as water (F) is partly or fully demineralized water, distilled or (multiply) redistilled water, water for medical or pharmaceutical purposes, for example purified water (Aqua purificata as per Pharm. Eur.).

The water (F) employed according to the invention preferably has a conductivity of less than 50 μS/cm, more preferably less than 10 μS/cm, and in particular less than 1.3 μS/cm, in each case at 25° C. and 1010 hPa.

The emulsions according to the invention preferably consist of
(A) amino-functional organopolysiloxanes containing Si—C-bonded radicals comprising basic nitrogen,
(C) inventive polyethylene glycol ethers of linear primary alcohols comprising linear hydrocarbon radicals having not more than 10 carbon atoms,
(E) optionally auxiliaries and
(F) water.

It is preferable when the emulsions according to the invention contain, preferably consist of,
10-60% by weight of amino-functional organopolysiloxanes (A)
0-10% by weight, preferably 0-2% by weight, and in particular 0-1% by weight, of organopolysiloxanes (B) which are distinct from (A),
5-30% by weight of nonionic emulsifiers (C) according to the invention
0-10% by weight of ionic emulsifiers (D)
0-5% by weight of auxiliaries (E) and
20-85% by weight of water (F),
based on the total weight of the emulsions.

It is particularly preferable when the emulsions according to the invention contain, and in particular are limited to,
25-45% by weight of amino-functional organopolysiloxanes (A),
0-5% by weight, preferably 0-2% by weight, and in particular 0-1% by weight, of organopolysiloxanes (B) which are distinct from (A),
10-20% by weight of nonionic emulsifiers (C) according to the invention
0-5% by weight of ionic emulsifiers (D)
0-2% by weight of auxiliaries (E) and
40-65% by weight of water (F),
based on the total weight of the emulsions.

In a preferred embodiment of the invention the emulsions according to the invention contain not less than 30% by weight and not more than 40% by weight of amino-functional organopolysiloxanes (A).

It is preferable when in the emulsions according to the invention the proportion of emulsifiers (C), (D) and auxiliaries (E) is altogether less than 50% by weight based on the total weight of the organopolysiloxanes (A) and optionally (B).

Production of the emulsions according to the invention is carried out according to processes that are known per se.

Production is typically carried out by simple stirring together of all constituents.

Suitable mixing and homogenizing apparatuses for producing the emulsions according to the invention include all emulsifying devices known to those skilled in the art, for example high-speed stirrers, dissolver disks, rotor-stator homogenizers, ultrasonic homogenizers and high-pressure homogenizers of any possible design. When large particles are desired, low-speed stirrers are also suitable.

The emulsions according to the invention may be produced on a continuous, semi-continuous or discontinuous basis.

Production of the emulsions according to the invention is preferably carried out at temperatures of 5° C. to 80° C., in particular 15° C. to 60° C., and preferably at the pressure of the ambient atmosphere. However, it is also possible to employ a vacuum or positive pressure.

It is preferable when initially the oils (A) and (B), the emulsifiers (C) and (D) and also the auxiliaries (E) and optionally not more than 20% of the total amount of water (F) are mixed with one another and homogenized. The water or remaining water is then mixed in slowly or portionwise.

The emulsions according to the invention are preferably clear to translucent and have turbidity values (at 90°) of preferably <500 ppm, more preferably <100 ppm, and most preferably of <20 ppm.

The emulsions according to the invention have a particle size (median of volume distribution D(50)) of <100 nm, preferably <50 nm, more preferably <10 nm.

The viscosity of the emulsions according to the invention at 25° C. is less than 10,000 mPas, preferably less than 5000 mPas, and most preferably less than 1000 mPas.

The emulsions according to the invention preferably have a pH of 4 to 8, more preferably 4 to 6.

Compared to the prior art, the emulsions according to the invention have the advantage that even at high silicon contents they exhibit a low viscosity and allow a good handleability without addition of viscosity-reducing additives, for example glycols, glycerol or salts, which may be disruptive in use.

Even with less than 50% emulsifiers and additives based on the silicone (A) and optionally (B), the emulsions according to the invention exhibit a low particle size, high transparency/clarity and excellent stability.

The emulsions according to the invention may be employed anywhere where emulsions based on polyorganosiloxanes having functional groups bearing a basic nitrogen have also hitherto been employed, for example as a textile softener, as an additive for washing compositions, as a release agent and in haircare compositions such as conditioner and shampoos.

The invention provides cosmetic formulations, in particular hair treatment compositions, containing the aqueous emulsions according to the invention.

The invention therefore further provides a process for care and cleaning of hair, wherein the hair is treated with the aqueous emulsions according to the invention or the cosmetic formulations according to the invention.

The invention further provides washing and cleaning compositions containing the aqueous emulsions according to the invention.

The invention therefore further provides a process for care and cleaning of fibers, in particular textile fibers and textile fabrics, wherein said fibers are treated with the aqueous emulsions according to the invention or the washing and cleaning compositions according to the invention.

The emulsions produced in the examples which follow were tested as follows:

Particle size was determined by dynamic light scattering using a Zetasizer ZEN1600/ Nano-S particle size analyzer from Malvern, Software Version 6.01. To this end the emulsions were diluted to 0.5% with filtered and degassed water. The reported values always relate to the D(50) value, this is the median of the volume distribution as described in Basic Principles of Particle Size Analysis, Technical Paper, Dr. Alan Rawle, Malvern Instruments Limited 2000. The following parameters were used: material RI 1.39, dispersant RI 1.33, material absorption 0.010, viscosity 0.8872 mPas, temperature 25° C., count rate 159.6, duration 50 s, measuring position 4.65 mm.

The turbidity of the emulsions was determined at an angle of 90°, also at 25° C., with a LabScat 2-angle turbidity meter from

Sigrist in a wide-necked glass bottle (250 mL, d=68 mm, h=115 mm).

The viscosities of the emulsions were determined according to DIN EN ISO 2555 using a “Brookfield programable Viscosimeter DV-II+” with spindle 3 to spindle 7 at 20° C. and 20 revolutions/min; the value was read off after 20 s.

Measurement of the pH was carried out according to US Pharmacopeia USP 33 at 20° C.

The amine number indicates how many mmol of amino groups are present per g of the siloxane A. The amine number may be determined by potentiometric titration with perchloric acid of the salts formed by protonation with glacial acetic acid.

The following substances were used:

Siloxane A1: a reactive (hydroxy/methoxy-terminal) aminoethylaminopropyl-functional polydimethylsiloxane having a viscosity of 1000 mPas at 25° C. and an amine number of 0.3 mmol/g, obtainable under the name Finish WR 1300 from Wacker Chemie AG, D-Munich

Siloxane A2: a trimethylsiloxy-terminated aminoethylaminopropyl-functional polydimethylsiloxane having a viscosity of 1000 mPas at 25° C. and an amine number of 0.6 mmol/g, obtainable under the name Finish WT 1650 from Wacker Chemie AG, D-Munich

Emulsifier C1 n-decylpolyethylene glycol ether having on average 6 ethylene glycol groups obtainable under the name Aduxol 10 D 06 from Chemische Fabrik Schärer & Schläpfer AG, CH-Rothrist

Emulsifier C2 n-decylpolyethylene glycol ether having on average 5 ethylene glycol groups obtainable under the name Aduxol 10D 05 from Chemische Fabrik Schärer & Schläpfer AG, CH-Rothrist

Emulsifier C3 n-hexylpolyethylene glycol ether having on average 5 ethylene glycol groups obtainable under the name Emulan HE 50 from BASF SE, D-Ludwigshafen.

Emulsifier C4: n-octylpolyethylene glycol ether having on average 4 ethylene glycol groups obtainable under the name Dehydol 04 DEO from BASF SE, D-Ludwigshafen.

Emulsifier CV1: (noninventive) iso-decylpolyethylene glycol ether having on average 6 ethylene glycol groups obtainable under the name Aduxol DEC 06 from Chemische Fabrik Schärer & Schläpfer AG, CH-Rothrist

Emulsifier CV2: (noninventive) iso-decylpolyethylene glycol ether having on average 5 ethylene glycol groups obtainable under the name LUTENSOL® XP 50 from BASF SE, D-Ludwigshafen

Emulsifier CV3: (noninventive) iso-tridecylpolyethylene glycol ether having on average 6 ethylene glycol groups obtainable under the name IMBENTIN® T060 from KOLB AG, CH-Hedingen

Emulsifier CV4: (noninventive) n-dodecylpolyethylene glycol ether having on average 4 ethylene glycol groups obtainable under the name SYMPATENS® ALM/040 from KOLB AG, CH-Hedingen

Emulsifier CV5: (noninventive) n-dodecylpolyethylene glycol ether having on average 23 ethylene glycol groups obtainable under the name SYMPATENS® ALM/230 G from KOLB AG, CH-Hedingen

Emulsifier D1: hexadecyltrimethylammonium chloride, obtainable as a 30% aqueous solution under the name Genamin° CTAC from Clariant GmbH, D-Frankfurt/Main

Emulsifier D2: cocoalkylbis(hydroxyethyl)methyl, ethoxylated, methylsulfate obtainable under the name SERVAMINE® KW 100 from Elementis Specialties Netherlands B.V., NL-Delden.

Auxiliary E1: Acetic acid Pharm. Eur., obtainable from CSC Jäklechemie, D-Nuremberg, for regulating pH

Auxiliary E2: Phenoxyethanol cosmetic quality, obtainable from Schülke&Mayr GmbH, D-Norderstedt, as a preservative

### EXAMPLE 1

122.5 g of siloxane A1, 49 g of emulsifier C1, 0.7 g of acetic acid (E1) and 3.15 g of phenoxyethanol (E2) are mixed with an ULTRA-TURRAX® T50 mixer for 1 min at 4000 rpm. Subsequently, 90 g of deionized water are added over 3 min and the mixture is homogenized at 4000 rpm for a further 5 min. The pasty emulsion formed was diluted with 85.1 g of water over 5 min and stored at 60° C. for 4 h. A clear, free-flowing emulsion was obtained. This emulsion was completely stable after 28 d at 50° C. and after storage for one year at room temperature.

### Comparative Example C1

Example 1 is repeated but using emulsifier CV 1 instead of emulsifier C1. A firm gel which was still not free-flowing even after 4 h at 60° C. is obtained.

### Comparative Example C2

The product from comparative example V1 is stored at 80° C. for 16 h. The mixture was now mobile but turbid. After 28 d at 50° C. the emulsion had separated.

### Comparative Example C3

Example 1 is repeated but using emulsifier CV 2 instead of emulsifier C1. A firm gel which was still very viscous even after 4 h at 60° C. is obtained.

### Comparative Example C4

Example 1 is repeated but using emulsifier CV 3 instead of emulsifier C1. A firm gel which was still very viscous even after 4 h at 60° C. is obtained.

### Comparative Example C5

Example 1 is repeated but using 32.66 g of emulsifier CV 4 and 16.33 g of emulsifier CV 5 instead of 49 g of emulsifier C1. A firm gel which was still firm even after 4 h at 60° C. is obtained.

### EXAMPLE 2

122.5 g of siloxane A2, 49 g of emulsifier C1, 0.7 g of acetic acid (E1) and 3.15 g of phenoxyethanol (E2) are mixed with an ULTRA-TURRAX® T50 mixer for 1 min at 4000 rpm. Subsequently, 80 g of deionized water are added over 3 min and the mixture is homogenized at 4000 rpm for a further 5 min. The pasty emulsion formed was diluted with 95.1 g of water over 5 min. A clear, low-viscosity emulsion was obtained.

### EXAMPLE 3

140 g of siloxane A1, 56 g of emulsifier C1, 4.0 g of emulsifier D1, 0.8 g of acetic acid (E1) and 3.6 g of phenoxyethanol (E2) are mixed with an ULTRA-TURRAX® T50 mixer for 1 min at 4000 rpm. The pasty emulsion formed was diluted with 145.6 g of water over 5 min. A clear, free-flowing emulsion was obtained.

### Comparative Example C6

Example 4 is repeated but using emulsifier CV 1 instead of emulsifier C1. A viscous emulsion which was still very viscous even after 4 h at 60° C. is obtained.

### EXAMPLE 4

122.5 g of siloxane A1, 49 g of emulsifier C1, 1.15 g of emulsifier D2, 0.7 g of acetic acid (E1) and 3.15 g of phenoxyethanol (E2) are mixed with an ULTRA-TURRAX® T50 mixer for 1 min at 4000 rpm. Subsequently, 173.5 g of deionized water are added over 3 min and the mixture is homogenized at 4000 rpm for a further 5 min. A clear, free-flowing emulsion was obtained.

### EXAMPLE 5

122.5 g of siloxane A1, 24.5 g of emulsifier C1, 24.5 g of emulsifier C3, 0.7 g of acetic acid (E1) and 3.15 g of phenoxyethanol (E2) are mixed with an ULTRA-TURRAX® T50 mixer for 1 min at 4000 rpm. Subsequently, 174.65 g of deionized water are added over 3 min and the mixture is homogenized at 4000 rpm for a further 5 min. An almost clear, mobile emulsion was obtained.

### EXAMPLE 6

122.5 g of siloxane A2, 24.5 g of emulsifier C2, 24.5 g of emulsifier C3, 0.7 g of acetic acid (E1) and 3.15 g of phenoxyethanol (E2) are mixed with an ULTRA-TURRAX® T50 mixer for 1 min at 4000 rpm. Subsequently, 174.65 g of deionized water are added over 3 min and the mixture is homogenized at 4000 rpm for a further 5 min. An almost clear, free-flowing emulsion was obtained.

### EXAMPLE 7

122.5 g of siloxane A1, 49.0 g of emulsifier C4, 3.5 g of emulsifier D1, 0.7 g of acetic acid (E1) and 3.15 g of phenoxyethanol (E2) are mixed with an ULTRA-TURRAX® T50 mixer for 1 min at 4000 rpm. Subsequently, 171.15 g of deionized water are added over 3 min and the mixture is homogenized at 4000 rpm for a further 5 min. An almost clear, mobile emulsion was obtained.

### EXAMPLE 8

122.5 g of siloxane A2, 49.0 g of emulsifier C2, 0.7 g of acetic acid (E1) and 3.15 g of phenoxyethanol (E2) are mixed with an ULTRA-TURRAX® T50 mixer for 1 min at 4000 rpm. Subsequently, 174.65 g of deionized water are added over 3 min and the mixture is homogenized at 4000 rpm for a further 5 min. An almost clear, mobile emulsion was obtained.

### Comparative Example C7

Example 9 is repeated but using emulsifier CV 2 instead of emulsifier C2. A turbid, viscous emulsion is obtained.

TABLE 1
Properties of the products obtained in the examples

| | Siloxane | | Viscosity | | Turbidity at |
|---|---|---|---|---|---|
| | proportion/emulsifiers | | in mPas | D(50) | 90° |
| Example | in % by weight | Appearance | at 25° C. | in nm | in ppm |
| 1 | 35/14 | clear | 1500 | 5.4 | 70.2 |
| C1 | 35/14 | firm gel | — | — | — |
| C2 | 35/14 | turbid | 970 | not | >1000 |
| | | | | measurable | |
| C3 | 35/14 | clear | 11680 | 3.3 | 16.4 |
| C4 | 35/14 | clear | 17080 | 3.6 | 21.9 |
| C5 | 35/14 | firm gel | — | — | — |
| 2 | 35/14 | almost clear | 360 | 35.0 | 68.6 |
| 3 | 40/16.34 | clear | 2190 | 10.3 | 53.3 |
| C6 | 40/16.34 | very viscous, | 15160 | 48.2 | 750 |
| | | turbid | | | |
| 4 | 35/14.3 | almost clear | 610 | 45.0 | 80.6 |
| 5 | 35/14 | clear | 980 | 5.9 | 48.9 |
| 6 | 35/14 | almost clear | 190 | 26.8 | 115 |
| 7 | 34/14.3 | almost clear | 130 | 36.5 | 106 |
| 8 | 34/14 | slightly turbid | 1010 | 10.4 | 365 |
| C7 | 34/14 | viscous, turbid | 430 | 37.5 | >1000 |

The noninventive emulsions are difficult to handle since, like C1 or C5, they afford a firm gel or, like C3, C4 and C6, are very viscous or, like C2, C6 and C7, are turbid.

By contrast, the emulsions according to the invention are readily handleable since they have a low viscosity and are clear and not turbid.

Use Example A1:

A conditioner is formulated as follows, the individual components being described according to the INCI nomenclature:

87.04 parts of water are initially charged and heated to 75° C. with stirring. In the course of this, 1.2 parts of hydroxyethylcellulose (obtainable under the name Tylose® H 4000 P2 from Shin-Etsu Chemical Co., J-Tokyo) are added. Once a temperature of 65° C. has been reached, 0.5 part of Stearamidopropyl Dimethylamine (obtainable under the name Incromine™ SB from Croda GmbH, D-Nettetal), 1 part of Polysorbate 80 (obtainable under the name Tween™ 80 from Croda GmbH, D-Nettetal), 3 parts of Stearyl Alcohol (obtainable under the name Stearyl Alcohol from Merck-Schuchardt, D-Hohenbrunn), 1 part of Cetyl Alcohol (obtainable under the name Cetyl

Alcohol from Merck KGaA, D-Grafing) and 1.76 parts of Behentrimonium Chloride (obtainable under the name Genamin® KDMP from Clariant GmbH, D-Frankfurt/Main) are added. The mixture is mixed until a temperature of 75° C. has been reached. The mixture is then cooled. During cooling, 0.2 part of Citric Acid (obtainable under the name Citric Acid from Sigma-Aldrich Chemie GmbH, D-Taufkirchen) and 0.2 part of Tetrasodium EDTA (obtainable under the name EDETA® B powder from BASF SE, D-Ludwigshafen) are added. Once a temperature of 35° C. has been reached, 0.1 part of preservative

Methylchloroisothiazolinone/Methylisothiazolinone (obtainable under the name MICORCARE® IT from Thor GmbH Speyer), 4 parts of a Dimethiconol emulsion (obtainable under the name BELSIL® DM 3112 VP from Wacker Chemie AG, Munich) and 2 parts of the emulsion from example 1 are added and the mixture is stirred for 5 minutes. The mixture is finally homogenized for one minute using an ULTRA-TURRAX® T50.

The thus obtained conditioner improves not only dry and wet combability but also improves feel in wet and in dry hair.

Use Example A2:

The emulsion of example 5 is diluted to a silicone content of 0.5%. A cotton fabric is immersed in this liquor and a liquid absorption of about 75% by weight based on the weight of the cotton is established by wringing. The fabric was subsequently dried for 5 min at 150° C. The fabric subsequently had an excellent soft hand.

1.-12. (canceled)

13. An aqueous emulsion, consisting of:
(A) 25-45% by weight of amino-functional organopolysiloxanes containing Si—C-bonded radicals containing basic nitrogen,
(B) 0-5% by weight of organopolysiloxanes which are distinct from the amino-functional organopolysiloxanes (A),
(C) 10-20% by weight of nonionic emulsifiers,
(D) 0-5% by weight of ionic emulsifiers,
(E) 0-2% by weight of auxiliaries and
(F) 40-65% by weight of water,
wherein as nonionic emulsifiers (C) polyethylene glycol ethers of linear primary alcohols comprising linear hydrocarbon radicals having not more than 10 carbon atoms, of formula (VI) are employed
CH_{3—}(CH₂)ₘ-13 O—(CH_{2—}CH_{2—}O)ₚ—R⁶ (VI),
wherein R⁶ is hydrogen or a C₁₋₄-hydrocarbon radical,
m represents an integer from 5 to 9 and
p is an integer from 3 to 20,
with the proviso that the co-use of
polyethylene glycol ethers of primary alcohols comprising branched hydrocarbon radicals and of
polyethylene glycol ethers of secondary alcohols are excluded.

14. The aqueous emulsion of claim 13, wherein the amino-functional organopolysiloxanes (A) are of the formula
R*_{3-y}Q_{y}SiO[R₂SiO]ₖ[RQSiO]ₗSiQ_{y}R*_{3-y} (III),
wherein
R* represents R or a radical of formula —OR²,
R is identical or different and represents a monovalent, optionally substituted hydrocarbon radical having 1 to 30 carbon atoms or hydrogen,
R² is identical or different and represents hydrogen or a monovalent, optionally substituted hydrocarbon radical having 1 to 20,
Q represents an amine-substituted radical of formula
—R³—[NR⁴—R⁵—]ₓNR⁴₂ (II)
in which
R³ is identical or different and represents a divalent Si—C-bonded hydrocarbon radical having 1 to 18 carbon atoms,
R⁴ represents hydrogen or a monovalent linear, cyclic or branched, saturated or unsaturated hydrocarbon radical having 1-18 carbon atoms,
R⁵ is identical or different and represents a divalent hydrocarbon radical having 1 to 6 carbon atoms,
x is 0 or an integer from 1 to 10,
k is an integer from 10 to 1000,
l is an integer from 1 to 100 and
y is 0 or 1, preferably 0.

15. The aqueous emulsion of claim 13, wherein cationic emulsifiers (D) are co-used.

16. The aqueous emulsion of claim 14, wherein cationic emulsifiers (D) are co-used.

17. The aqueous emulsion of claim 13, wherein at least one y auxiliary (E) selected from the group consisting of preservatives, pH regulators, silanes, rheology additives, disinfectants, wetting agents, corrosion inhibitors, colorants or fragrances, are present.

18. In a process for preparing a cosmetic formulation, where an aqueous emulsion of an organopolysiloxane is employed, the improvement comprising employing an aqueous emulsion of claim 13 as the aqueous emulsion of an organopolysiloxane.

19. In a washing and/or cleaning composition wherein an aqueous emulsion of an organopolysiloxane is employed, the improvement comprising employing an aqueous emulsion of claim 13 as the aqueous emulsion of an organopolysiloxane.
